(19) United States
(12) Patent Application Publication
(10) Pub. No.: US 2011/0034498 A1
(43) Pub. Date: Feb. 10, 2011

(54) DOSING REGIMENS FOR THE TREATMENT OF CANCER

(76) Inventors: Karen J. Mcgovern, Littleton, MA (US); Christine S. Pien, Newbury, MA (US); James L. Wright, Lexington, MA (US)

Correspondence Address: MORRISON & FOERSTER LLP, 12531 HIGH BLUFF DRIVE, SUITE 100, SAN DIEGO, CA, 92130-2040, US

(21) Appl. No.: 12/293,524

(22) PCT Filed: Mar. 24, 2006
(86) PCT No.: PCT/US06/10796

§ 371 Date: Aug. 24, 2010

(51) Int. Cl. A61K 31/4355; A61P 35/00

(52) U.S. Cl. 514/278

(57) ABSTRACT

The invention relates to a method of treating cancer. The method includes administering systemically a therapeutically effective amount of a small molecule hedgehog pathway inhibitor, such that the concentration of the inhibitor in the blood does not vary by more than about ±30% from the average concentration, and such that the concentration remains at or below the maximum tolerated dose of the inhibitor for a time period of at least about one day.

### BACKGROUND OF THE INVENTION

The present invention relates to dosing regimens for the treatment of cancers that are dependent, at least in part, on the hedgehog pathway for survival.

Inhibition of the hedgehog pathway in certain cancers has been shown to result in inhibition of tumor growth. For example, anti-hedgehog antibodies have been shown to antagonize the function of the hedgehog pathway and inhibit the growth of tumors. Small molecule inhibition of hedgehog pathway activity has also been shown to result in cell death in a number of cancer types.

Research in this area has focused primarily on the elucidation of hedgehog pathway biology and the discovery of new hedgehog pathway inhibitors. Although potent inhibitors of the hedgehog pathway have been identified, progress toward the development of clinical candidates has been hampered due to a poor understanding of the dosing regimen required to optimally treat hedgehog pathway mediated diseases.

### SUMMARY OF THE INVENTION

The present invention relates to a method of treating cancer in a human in need of such treatment. The method includes administering systemically to the human a therapeutically effective amount of a small molecule hedgehog pathway inhibitor, where the concentration of the inhibitor in the blood of the human does not vary by more than about ±30% from the average concentration, and remains at or below the maximum tolerated dose for a time period of at least about one day.

The inhibitor can be a steroidal alkaloid. The inhibitor can be administered, e.g., intramuscularly, intravenously, subcutaneously, or orally, e.g., in a sustained release pill. The inhibitor can also be administered using continuous infusion, or a sustained release device, e.g., a pump, a biodegradable polymer, or a non-biodegradable polymer.

The time period specified above can also be at least about three days, at least about one week, at least about two weeks, or at least about one month. In addition, in some embodiments, the concentration of the small molecule hedgehog pathway inhibitor in the blood of the human does not vary by more than about ±20%, or about ±15%.

The present invention also relates to a method of treating cancer in a human in need of such treatment, where the method includes administering systemically to the human a therapeutically effective amount of a small molecule hedgehog pathway inhibitor, where the inhibitor is present in the blood of the human at a concentration that is maintained above a threshold concentration, and remains at or below the maximum tolerated dose for a time period of at least about one day.

The small molecule hedgehog pathway inhibitor can be a steroidal alkaloid. The inhibitor can be administered, e.g., intramuscularly, intravenously, subcutaneously, or orally, e.g., in a sustained release pill. The inhibitor can also be administered using continuous infusion or a sustained release device, e.g., a pump, a biodegradable polymer, or a non-biodegradable polymer.

In some embodiments, the inhibitor can be present in the blood at a concentration of about two times, about five times, about ten times, or about twenty times the threshold concentration. In addition, the time period specified above can be at least about three days, at least about one week, at least about two weeks, or at least about one month.

The small molecule hedgehog pathway inhibitor can be selected from the group consisting of

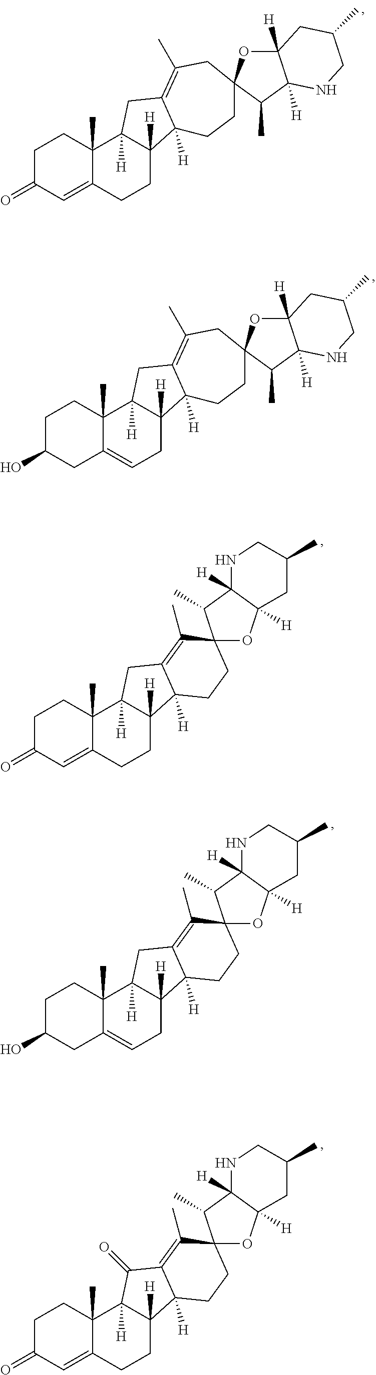

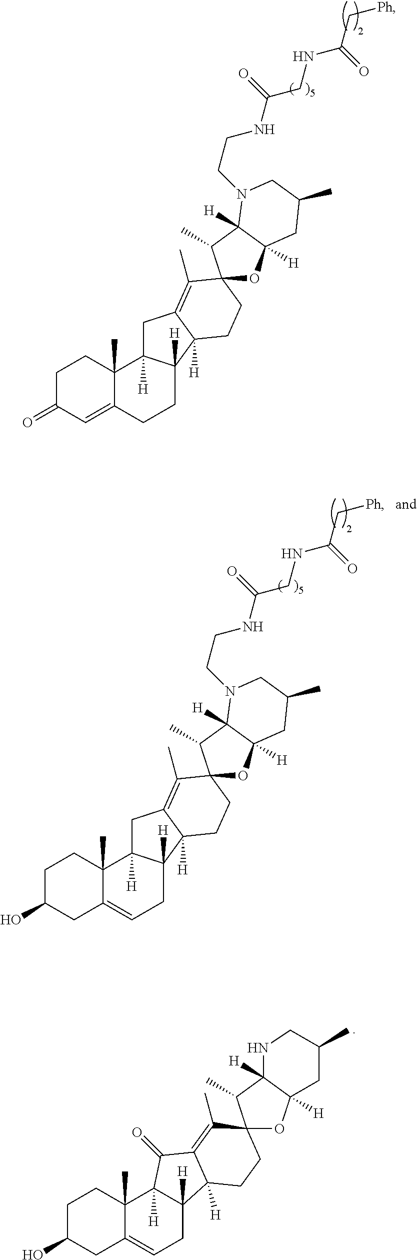

### BRIEF DESCRIPTION OF THE FIGURES

FIG. 1 depicts the blood concentration versus time of an exemplary hedgehog pathway inhibitor when dosed subcutaneously twice a day (18.7 mg/dose).

FIG. 2 depicts the blood concentration versus time of an exemplary hedgehog pathway inhibitor when dosed orally in an instant release form four times a day (31.2 mg/dose).

FIG. 3 depicts the blood concentration versus time of an exemplary hedgehog pathway inhibitor when dosed orally in sustained release form twice a day (62.4 mg/dose).

FIG. 4 depicts the blood concentration versus time of an exemplary hedgehog pathway inhibitor when dosed subcutaneously in sustained release form once a day (83.2 mg/dose).

### DETAILED DESCRIPTION OF THE INVENTION

The present invention generally relates to dosing regimens for the treatment of disorders characterized by uncontrolled hedgehog pathway activation, such as cancer. The following definitions apply to the terms as they are used throughout this specification, unless otherwise limited in specific instances, either individually or as part of a larger group.

An “effective amount” of a compound refers to an amount in a preparation which, when applied as part of a desired dosage regimen, brings about a change in the rate of cell proliferation and/or rate of survival of a cell according to clinically acceptable standards for the disorder to be treated.

The term “hedgehog pathway antagonist” refers to an agent that binds to and inhibits the function of the hedgehog protein and/or smoothened (Smo), or binds to and agonizes the function of patched (Ptch). Such binding results in inhibition of the function of the hedgehog pathway.

The phrase “inhibition of tumor growth” as used herein means causing a reduction in or complete cessation of tumor growth and/or causing a regression in tumor size.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “proliferating” and “proliferation” refer to cells undergoing mitosis.

The phrase “therapeutically-effective amount” as used herein means that amount of a compound, material, or composition which is effective for producing some desired therapeutic effect in at least a sub-population of cells in an animal.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and infrasternal injection and infusion.

The phrase “threshold concentration” as used herein is the lowest concentration of a drug in a human at which a desired therapeutic effect is observed (e.g., inhibition of tumor growth).

The phrases “systemic administration,” “administered systemically,” “peripheral administration,” and “administered peripherally” as used herein mean the administration of a compound, drug or other material, other than directly into the site of the hedgehog mediated disease, such that it enters the patient's system and, thus, is subject to metabolism and other like processes. An example of systemic administration is subcutaneous administration.

The phrase “sustained release” is used in its conventional sense to refer to a drug formulation that provides for gradual release of a drug over an extended period of time, and that preferably, although not necessarily, results in substantially constant blood levels of a drug over a period of time, such as, at least about one month, about three weeks, about two weeks, about one week, about 3 days, about 24 hours, about 18 hours, about 12 hours, about 10 hours, about 8 hours, about 7 hours, about 6 hours, or about 5 hours after drug administration.

When inhibitors are dosed according to the methods of the present invention, the efficacy of a hedgehog inhibitor can be improved, toxic side effects of the inhibitor can be decreased, and/or the maximum tolerated dose for the hedgehog pathway inhibitor can be increased, as compared to using traditional dosing regimens. An example of a traditional dosing regimen in one in which the inhibitor is dosed at the maximum tolerated dose followed by a recovery period, where the concentration of the inhibitor in the blood of the subject is allowed to vary substantially, and/or is allowed fall below the threshold concentration for the inhibitor.

Hedgehog pathway inhibitors useful in the methods of the present invention generally act at or downstream of the position in the hedgehog pathway that is associated with the elevated hedgehog pathway activity. For example, where elevated hedgehog pathway activity is ligand stimulated, the hedgehog antagonist can be selected based on the ability, for example, to sequester the hedgehog ligand or to reduce or inhibit binding of the hedgehog ligand to its receptor. Where elevated Hh pathway activity is due to an inactivating mutation of the hedgehog ligand receptor (e.g., Ptch), the Hh pathway antagonist can be selected based on the ability to bind to and inhibit Smo. Hedgehog pathway inhibitors are further exemplified by Smo antagonists such as steroidal alkaloids and derivatives thereof, including, for example, cyclopamine and derivatives thereof (see, e.g., Chen, et al., Genes Devel. (2002) 16:2743-2748; U.S. Pat. No. 6,432,970 B2, U.S. patent application Ser. No. 11/213,534; U.S. Pat. No. 6,613,798; U.S. Published Patent Application 2004/0023949) each of which is incorporated herein by reference), and SANT-1, SANT-2, SANT-3, and SANT-4 (see Chen, et al., Proc. Natl. Acad. Sci., USA (2002) 99:14071-14076, which is incorporated herein by reference).

Useful inhibitors may contain a basic functional group, such as an amino group, and are thus capable of forming pharmaceutically-acceptable salts with appropriate acids. The term “pharmaceutically-acceptable salts” in this respect, refers to the relatively non-toxic, inorganic and organic acid addition salts of inhibitors used in the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the bromide, chloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge, et al. “Pharmaceutical Salts”, J. Pharm. Sci. (1977) 66:1-19).

In other cases, the inhibitors may contain one or more acidic functional groups and, thus, are capable of forming pharmaceutically-acceptable salts with the appropriate bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge, et al., supra).

Based on the pharmacokinetics (e.g., rate of excretion, rate of absorption and order of absorption) of the hedgehog pathway inhibitor employed in the methods of the present invention, one of skill in the art can readily develop a dosing schedule and drug formulation to dose the inhibitor to the patient such that the concentration of the inhibitor in the blood of the patient remains substantially constant and/or such that the blood concentration of the inhibitor is maintained above a threshold concentration over a period of time. Depending on the absorption kinetics of the drug (i.e., first or second order absorption) blood concentration of the drug as a function of time since the last dose can be predicted. (See, Rowland, M., and Tozer, T. N., Clinical Pharmacokinetics, Lippincott Williams & Wilkins, 1995, herein incorporated by reference). For example, assuming first order absorption of the inhibitor, equation 1 can be used to predict the concentration of the inhibitor in the blood of the patient at time t.

C=FD/V(kₐ/(kₐ−k))(e^{-kt}−e^{-kat}) (1)
Where:
F=bioavailability D=dose (mg); V=volume of distribution (L);
T=time post dose (hr); k=elimination rate constant (hr⁻¹); and
kₐ=absorption rate constant (hr⁻¹).

By adjusting the rate of release of the drug (by selection of the formulation), the frequency of dosing of the drug (number of doses per day, week, or month) and the dose administered, the peak and trough blood levels of the drug can be exquisitely controlled, and as a result the blood concentration of the inhibitor can be controlled such that the concentration of the inhibitor does not vary from the average concentration of the inhibitor by more than ±30% over a period of time and/or the concentration of the inhibitor is maintained above a threshold concentration for a period of time.

As shown in the examples below, blood concentrations of hedgehog pathway inhibitors can be controlled such that the concentration of the inhibitor does not vary by more than about ±30%, or such that the blood concentration of the inhibitor is maintained above a threshold concentration. When the inhibitor is administered in this fashion, efficacy can be improved.

The hedgehog pathway inhibitor may be administered to a patient such that the blood concentration of the inhibitor in the patient remains substantially constant (e.g., does not vary by more than ±30% from the average blood concentration of the inhibitor) over a period of time (the period of time may be about at least about one day, two days, three days, four days, five days, six days, one week, two weeks, three weeks, four weeks, or two months). The inhibitor may also be administered such that the blood concentration of the inhibitor is maintained above the threshold concentration for the inhibitor being dosed, over a period of time, which may be at least about one day, two days, three days, four days, five days, six days, one week, two weeks, three weeks, four weeks, or two months).

The hedgehog pathway inhibitor may be administered to a patient such that the concentration of the inhibitor in the blood of the patient varies by no more than about ±30% from the average concentration of the inhibitor in the patient's blood over a period of time, no more than about ±20% from the average concentration of the inhibitor in the patient's blood over a period of dine, no more than about ±15% from the average concentration of the inhibitor in the patient's blood over a period of time, no more than about ±10% from the average concentration of the inhibitor in the patient's blood over a period of time, or no more than about ±7.5% from the average concentration of the inhibitor in the patient's blood over a period of time. For example, if the average concentration of the hedgehog pathway inhibitor in the blood of a patient is 10 ng/mL over a period time, then the concentration ranges of the inhibitor would be maintained between about 13 ng/mL to about 7 ng/mL (representing a change of no more than about ±30% from the average concentration), between about 12 ng/mL to about 8 nn/mL (representing a change of no more than about ±20% from the average concentration), between about 11.5 ng/mL to about 8.5 ng/mL (representing a change of no more than about ±15% from the average concentration), between 11 ng/mL to about 9 ng/mL (representing a change of no more than about ±10% from the average concentration), or between about 10.75 ng/mL to about 9.25 ng/mL (representing a change of no more that about ±7.5% from the average concentration).

The hedgehog pathway inhibitor may also be administered to a patient such that the concentration of the inhibitor in the blood of the patient remains at or above the threshold concentration and below the maximum tolerated dose of the inhibitor for a period of time. The inhibitor my be delivered to the patient such that the concentration of the inhibitor in the blood of the patient remains at or above about 1 times, about 1.1 times, about 1.5 times, about 2.0 times, about 2.5 times, about 3.0 times, about 3.5 times, about 4.0 times, about 4.5 times, about 5.0 times, about 6 times, about 7 times, about 8 times, about 9 times, about 10 times, about 15 times, or about 20 times the threshold concentration. For example, if the threshold concentration for a hedgehog pathway inhibitor is found to be 10 ng/mL, then the concentration of the inhibitor in the blood would not be allowed to drop below 10 ng/mL (representing a blood concentration of 1 times the threshold concentration), 11 ng/mL (representing a blood concentration of 1.1 times the threshold concentration), or 15 ng/mL (representing a blood concentration of 1.5 times the threshold concentration).

Cancers or neoplastic diseases and related disorders that can be treated by administration of a hedgehog pathway inhibitor employing methods of the present invention, include, but are not limited to, adrenal cortical cancer, anal cancer, aplastic anemia, basal cell carcinoma, bile duct cancer, bladder cancer, bone cancer, brain/CNS tumors, breast cancer, cervical cancer, lymphomas, colon cancer, rectum cancer, endometrial cancer, esophagus cancer, Ewing's family of tumors, eye cancer, gallbladder cancer, gastrointestinal tumors, gastrointestinal stromal tumors, gestational trophoblastic disease, hepatocellular cancer, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, small cell lung cancer, non-small cell lung cancer, lung carcinoid tumor, non-Hodgkin's type lymphoma, male breast cancer, malignant mesothelioma, medulloblastoma, multiple myeloma, myelodysplastic syndrome, nasal cavity and paranasal cancer, glioma, nasopharyngeal cancer, neuroblastoma, oral cavity cancer, oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, melanoma skin cancer, non-melanoma skin cancer, stomach cancer, testicular cancer, thymus cancer, thyroid cancer, uterine sarcoma, vaginal cancer, vulvar cancer, Waldenstrom's macroglobulinemia, and Wilms' tumor.

The methods of the present invention may also be used to treat other disorders, such as psoriasis.

Based on the properties (e.g., the rate of excretion, bioavailability, maximum tolerated dose, efficacy, etc.) of the hedgehog pathway inhibitor being dosed to the subject, the inhibitor may be formulated for delivery and/or dosed in such a way to provide either substantially constant blood levels of the inhibitor in the subject over a period of time or in a fashion such that the concentration of the inhibitor is maintained above a therapeutic threshold concentration for a period of time. In certain embodiments the blood concentration of the inhibitor may be controlled by formulating the inhibitor in sustain released form (e.g., a sustained release tablet for oral administration, or a suspension for subcutaneous, intraperitoneal, or intramuscular administration), dosing the inhibitor in a continuous fashion (e.g., continuous infusion), or administered using a sustained release device (e.g., a pump or polymer (biodegradable or non-biodegradable)). A comprehensive list of different techniques employed by those skilled in the art for delivering inhibitors according to the methods of the present invention can be found in Saltzman, W. M.; Drug Delivery; Engineering Principles for Drug Therapy, Oxford University Press, 2001 and Rathbone, M. J., et al., Modified-Release Drug Delivery Technology, Marcel Dekker, Inc., 2003 (both of which are incorporated herein by reference).

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a hedgehog pathway inhibitor.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form.

Injectable depot forms may be made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In certain embodiments, a suitable daily dose of a compound of the invention will be that amount of the compound which is the lowest dose effective to produce a therapeutic effect. Such an effective dose will generally depend upon the factors described above. Generally, oral, intravenous, intraperitoneal and subcutaneous doses of the compounds of this invention for a patient, when used for the indicated analgesic effects, will range from about 1 to about 1000 mg per m² of body surface area per day, from about 1 to about 100 mg per m² of body surface area per day, from about 10 to about 100 mg per m² of body surface area per day, or from about 10 to about 50 mg per m² of body surface area per day.

If desired, the effective daily dose of the active compound may be administered as two, three, four, five, six or more sub-doses administered separately at appropriate intervals throughout the day, optionally, in unit dosage forms. Alternatively, the daily dose can be administered once per day.

As described in detail below, the inhibitors useful in the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) intravaginally or intrarectally, for example, as a pessary, cream or foam; (4) sublingually; (5) ocularly; (6) nasally; (7) pulmonary; or (8) intrathecally.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient.

In solid dosage forms for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the compositions may also contain buffering agents. Solid compositions of a similar type may also be employed as fillers in soft and hard-shelled gelatin capsules using such excipients as lactose or milk sugars, as well as high molecular weight polyethylene glycols and the like.

The tablets, and other solid dosage forms of inhibitors useful in the methods of the present invention, such as dragees, capsules, pills and granules, may optionally be scored or prepared with coatings and shells, such as enteric coatings and other coatings. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds useful in the methods of the present invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Pharmaceutical compositions useful for parenteral administration can contain the inhibitor, in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use. These solutions may contain sugars, alcohols, antioxidants, buffers, bacteriostats, solutes which render the formulation isotonic with the blood of the intended recipient.

Formulations suitable for oral, nasal, (including buccal and sublingual), rectal, vaginal and/or parenteral administration may be presented in unit dosage form and may be prepared by any method well known in the art of pharmacy. The amount of active ingredient which can be combined with a carrier material to produce a single dosage form will vary depending upon the host being treated, and the particular mode of administration.

Formulations for rectal or vaginal administration may be presented as suppositories, which may be prepared by mixing inhibitors with one or more suitable nonirritating excipients or carriers containing, for example, cocoa butter, polyethylene glycol, suppository wax, or other substance which is solid at room temperature, but liquid at body temperature and, therefore, will melt in the rectum or vaginal cavity and release the active compound.

Formulations useful in the methods of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of this invention.

Powders and sprays can contain, in addition to hedgehog pathway inhibitors, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

### EXAMPLES

The invention now being generally described, it will be more readily understood by reference to the following examples, which are included merely for purposes of illustration of certain aspects and embodiments of the present invention, and are not intended to limit the invention.

### Example 1

### Synthesis of an Analog of Cyclopamine

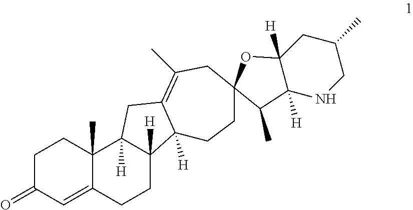

### Part A

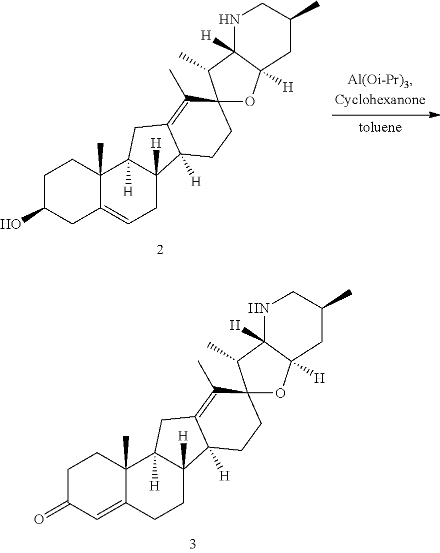

Cyclopamine 2 (20 mg, 0.049 mmol) was suspended in dry toluene (0.6 mL) and cyclohexanone (150 μL, 1.47 mmol, 30 eq), followed by aluminum isopropoxide (79 mg, 0.392 mmol, 8 eq). The resulting mixture was heated to reflux for 2 hours, cooled to room temperature, diluted with ethyl acetate and quenched with Rochelle's salt solution. The biphasic mixture was stirred overnight, the layers were separated, the aqueous layer was extracted with ethyl acetate, and the combined organic extracts were dried (over mgSO₄), filtered and concentrated in vacuo. The residue was purified by flash chromatography (DCM, DCM/methanol 98:2 and 95:5). Compound 3 was obtained as a white crystalline solid (70% yield).

### Part B

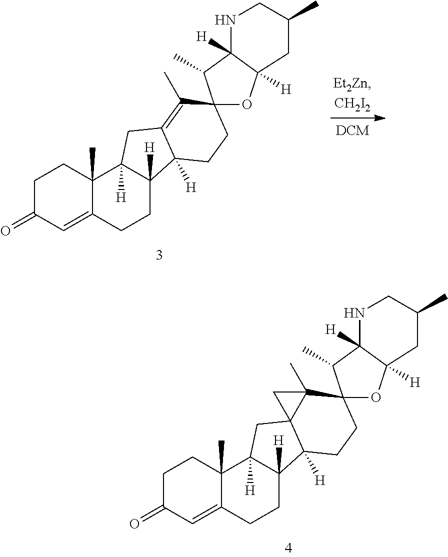

Diiodomethane (40 μl, 0.5 mmol, 2.5 eq) in DCM (0.52 mL) at 0° C. was treated with 15% diethylzinc in toluene (0.2 mL, 0.2 mmol 1 eq), and the resulting solution was stirred for 5 minutes, at which point a white precipitate was observed. The enone 3 (10 mg, 0.02 mmol, 1 eq) in DCM (0.35 mL) was added and the resulting mixture was stirred at room temperature (ice bath removed) for 3 hours, quenched with NaOH (2 N) and stirred for 10 minutes. The layers were separated and the aqueous layer was extracted with DCM (two times). The organic extracts were dried over mgSO₄, filtered and concentrated in vacuo. The residue was purified by flash chromatography (DCM/methanol 92:8). The cyclopropanated material 4 was obtained as a white solid.

### Part C

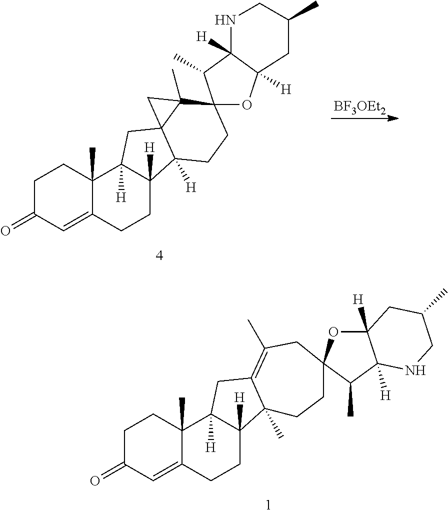

To a solution of cyclopropylenone 4 (10 mg, 24 μmol. 1 eq) in DCM (0.5 ml) at 0° C. under argon was added BF₃.Et₂O (30 μL, 0.24 mmol, 10 eq). The resulting solution was stirred at 0° C. for 1.5 hours, diluted with DCM, and quenched with saturated sodium bicarbonate. The organic phase was washed with saturated sodium bicarbonate, and the combined aqueous layers were extracted with DCM. The combined organic layers were washed with brine, dried over mgSO₄, filtered and concentrated in vacuo. The residue was purified by preparative TLC (DCM/methanol 9:1). Compound 1 was obtained as a white solid (90% yield). MS (ESI(+)) m/e 424.62 (M+H)⁺.

### Example 2

A study was performed in mouse PC-3 prostate xenograft models to assess the ability of the methods of the present invention to reduce subcutaneous tumor burden. In the study male athymic nude (Nu/Nu) mice were implanted with PC-3 cells (1×10⁷ cells) into the flank of the left leg. When the average tumor size reached 200 mm³ animals were randomly assigned to treatment groups (N=12/group). Mice were implanted with Alzet mini pumps containing either the HCl salt of compound 1 or vehicle (30% HPBCD in WFI). The mini pumps were surgically implanted subcutaneously on the flank of the right leg, contralateral to the site of the tumor implant. The pumps were replaced with new pumps every 6^{th} day of the study. In the study, mice received either vehicle or the test compound at 10.6 mg/kg/day. Tumor volumes for each group were measured at regular intervals during treatment. The animals were sacrificed after 25 days of treatment and tumor volumes were compared. The results of this experiment are summarized below. As shown, when mice were treated with continuous doses of the hedgehog antagonist, the growth of the tumors was significantly reduced.

| Group | Start Volume(mm³) | End Volume (mm³) |
|---|---|---|
| Vehicle | 234 ± 20 | 1238 ± 143 |
| Compound 1 | 188 ± 17 | 444 ± 101 |
| (10 mg/kg/day) | | |

Blood concentration of the compound 1 was monitored over time in three mice The average blood concentration of the three mice over 25 days is shown below.

| | Day | Day | Day | | Day | | |
|---|---|---|---|---|---|---|---|
| | 1 | 5 | 7 | Day 11 | 13 | Day 17 | Day 25 |
| Concentration | 0.096 | 0.119 | 0.132 | 0.0650 | 0.127 | 0.080 | 0.091 |
| (ug/mL) | | | | | | | |

### Example 3

A study was performed in mouse SKOV-3 xenograft models to assess the ability of the methods of the present invention to reduce subcutaneous tumor burden. Male athymic nude (Nu/Nu) mice were implanted with SKOV-3 cells (1×10⁷ cells) into the flank of the left leg. When the average tumor size reached 50 mm³, animals were randomly assigned to treatment groups. Mice were implanted with Alzet mini pump containing the HCl salt of compound 1 or vehicle (30% HPBCD in WFI). The mini pumps were surgically implanted subcutaneously on the flank of the right leg, contralateral to the site of the tumor implant. The pumps were replaced with new pumps every 6^{th} day of the study. Mice received either vehicle (N=13/group), or the test compound at 10.6 mg/kg/day (N=13/group) or 20 mg/kg/day (N=5/group). Tumor volumes of the two groups were measured at regular intervals during treatment, which lasted 74 days. The results of this experiment are summarized below. As shown, when mice were treated with continuous doses of the hedgehog antagonist, the growth of the tumors was significantly reduced.

| Group | Start Volume(mm³) | End Volume (mm³) |
|---|---|---|
| Vehicle | 58 ± 4 | 358 ± 52 |
| Compound 1 | 57 ± 5 | 186 ± 57 |
| (10.6 mg/kg/day) | | |
| Compound 1 | 31 ± 4 | 52 ± 11 |
| (20 mg/kg/day) | | |

### Example 4

A study was performed in mouse PAN-02 xenograft models to assess the ability of the methods of the present invention to reduce subcutaneous tumor burden. The PAN-02 cells were maintained by in vivo passaging in C57/1316 mice. Male C57/B16 mice were implanted with PAN-02 cells (8 mm³ tumor fragments) into the inguinal area. Each mouse was implanted with an 8 mm³ tumor PAN-02 tumor fragment collected from a donor mouse. The tumor fragment was loaded onto a 16 G trocar and implanted by piercing through the skin in the right inguinal area. The animals were randomly assigned to treatment groups (N=14/group). Mice were implanted with an Alzet mini pump containing the HC1 salt of compound 1, or vehicle (30% HPBCD in WFI). The mini pumps were surgically implanted subcutaneously on the flank of the right leg, contralateral to the site of the tumor implant. The pumps were replaced with new pumps every 6^{th} day of the study. Mice received either vehicle or the test compound at 20 mg/kg/day. Tumor volumes of the two groups were measured at regular intervals during treatment, which lasted 32 days. The results of this experiment are summarized below. As shown, when mice were treated with continuous doses of the hedgehog antagonist, the growth of the tumors was significantly reduced.

| Group | Start Volume(mm³) | End Volume (mm³) |
|---|---|---|
| Vehicle | 86 ± 9 | 838 ± 102 |
| Compound 1 | 82 ± 8 | 481 ± 64 |
| (20 mg/kg/day) | | |

### Example 5

A study was performed in mouse PC-3 prostate xenograft models to assess the ability of the methods of the present invention to reduce subcutaneous tumor burden. Male athymic nude (Nu/Nu) mice were implanted with PC-3 cells (1×10⁷ cells) into the flank of the left leg. When the average tumor size reached 200 mm³, animals were randomly assigned to treatment groups (N=12/group). Mice were implanted with Alzet mini pumps containing either the HC1 salt of cyclopamine, or vehicle (30% HPBCD in WFI). The mini pumps were surgically implanted subcutaneously on the flank of the right leg, contralateral to the site of the tumor implant. The pumps were replaced with new pumps every 6^{th} day of the study. Mice received either vehicle or the test compound at 10.6 mg/kg/day. Tumor volumes for each group were measured at regular intervals during treatment. The animals were sacrificed after 25 days of treatment, and tumor volumes were compared. The results of this experiment are summarized below. As shown, when mice were treated with continuous doses of the hedgehog antagonist, the growth of the tumors was significantly reduced.

| Group | Start Volume(mm³) | End Volume (mm³) |
|---|---|---|
| Vehicle | 168 ± 8 | 806 ± 155 |
| Cyclopamine | 170 ± 29 | 448 ± 152 |
| (10 mg/kg/day) | | |

### Example 6

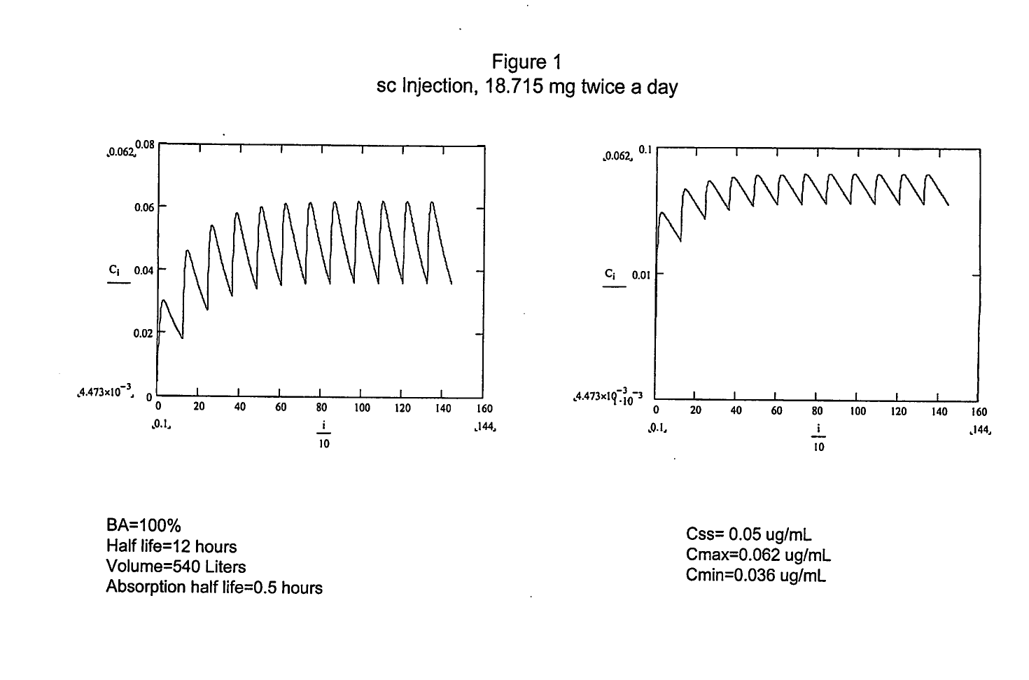

FIG. 1 depicts the blood concentration versus time of a hedgehog pathway inhibitor if dosed subcutaneously twice a day (18.7 mg/dose) in a patient. The figure illustrates how, using equation 1 (or equations for compounds with non-first order absorption rates), blood concentrations of the inhibitor can be controlled such that they do not vary by more than ±30%. In this example, bioavailability=100%; the half life of inhibitor=12 hours; the volume of distribution=540 Liters; and the absorption half life of inhibitor=0.5 hours.

### Example 7

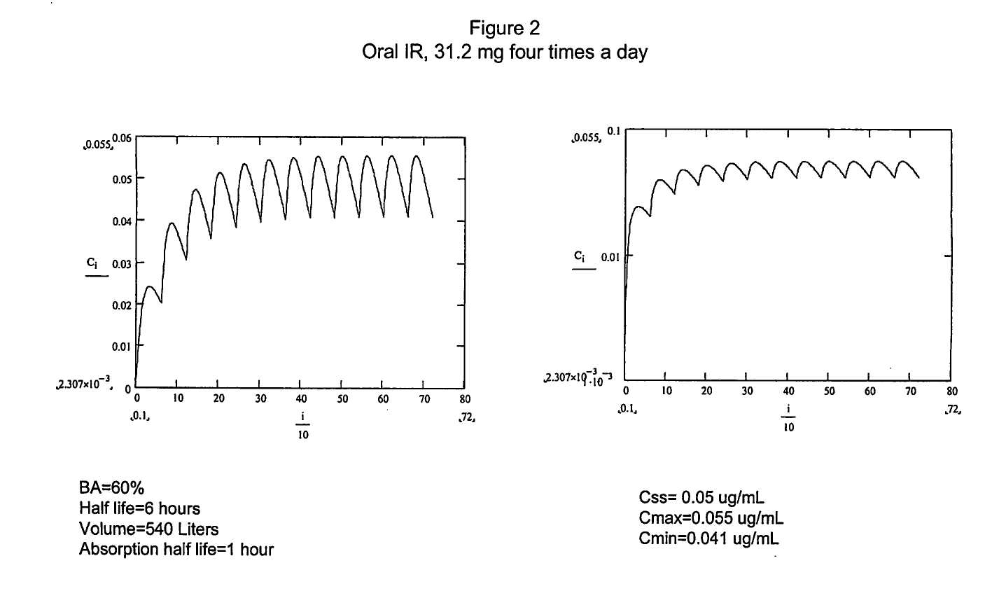

FIG. 2 depicts the blood concentration versus time of a hedgehog pathway inhibitor if dosed orally in an instant release form four times a day (31.2 mg/dose) in a patient. The figure illustrates how, using equation 1 (or equations for compounds with non-first order absorption rates), blood concentrations of the inhibitor can be controlled such that they do not vary by more than ±20%. In this example, bioavailability=60%; the half life of the inhibitor=6 hours; the volume of distribution=540 Liters; the absorption half life of the inhibitor=1 hour.

### Example 8

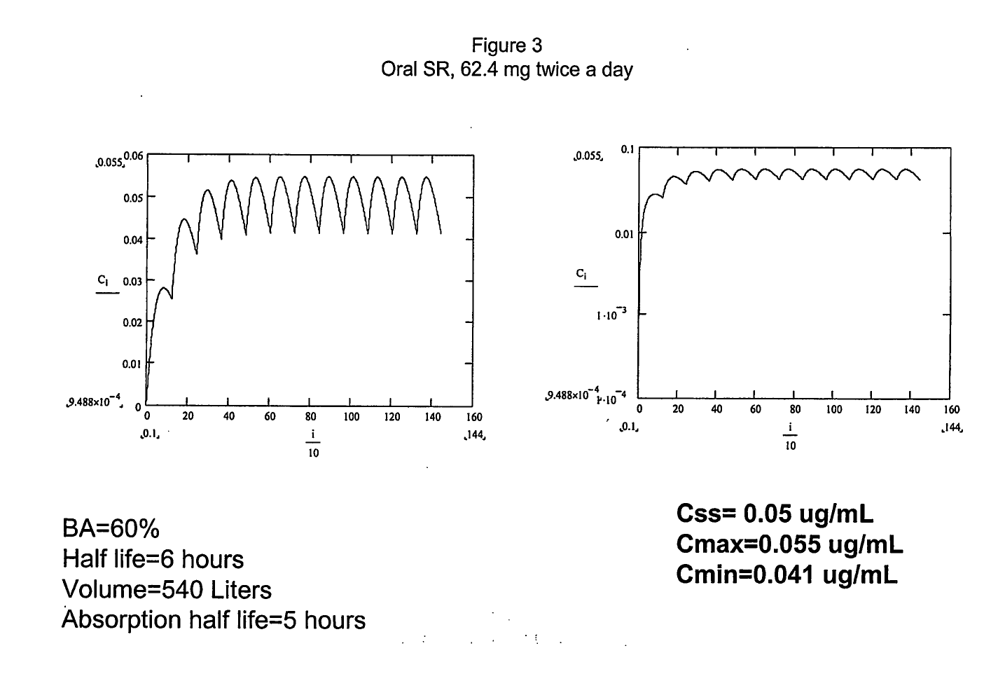

FIG. 3 depicts the blood concentration versus time of a hedgehog pathway inhibitor if dosed orally in sustained release form twice a day (62.4 mg/dose) in a patient. The figure illustrates how, using equation 1 (or equations for compounds with non-first order absorption rates), blood concentrations of the inhibitor can be controlled such that they do not vary by more than ±20%. In this example, bioavailability=60%; the half life of the inhibitor=6 hours; the volume of distribution=540 Liters; and the absorption half life of the inhibitor=5 hours.

### Example 9

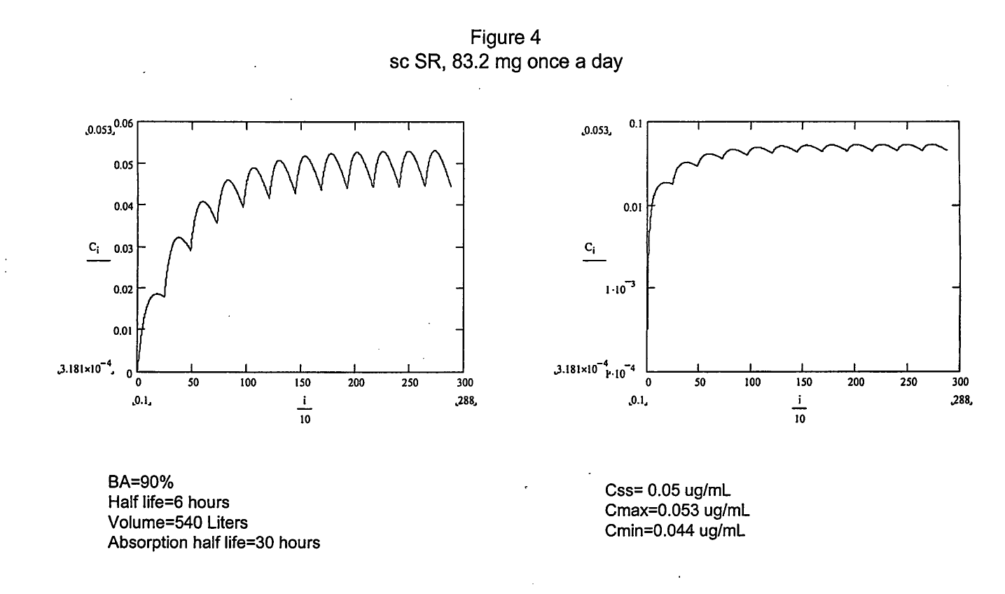

FIG. 4 depicts the blood concentration versus time of a hedgehog pathway inhibitor if dosed subcutaneously in sustained release form once a day (83.2 mg/dose) in patient. The figure illustrates how, using equation 1 (or equations for compounds with non-first order absorption rates), blood concentrations of the inhibitor can be controlled such that they do not vary by more than ±15%. In this example, bioavailability=90%; the half life of the inhibitor=6 hours; the volume of distribution=540 Liters; and the absorption half life of the inhibitor=305 hours.

### INCORPORATION BY REFERENCE

All references, including all U.S. patents and U.S. published patent applications, cited herein are hereby incorporated by reference in their entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating cancer in a human in need of such treatment, comprising administering systemically to said human a therapeutically effective amount of a small molecule hedgehog pathway inhibitor, wherein the concentration of said inhibitor in the blood of said human does not vary by more than about ±30% from the average concentration of said inhibitor in the blood of said human, and said concentration remains at or below the maximum tolerated dose of said inhibitor for a time period of at least about one day.

2. The method of claim 1, wherein said inhibitor is a steroidal alkaloid.

3. The method of claim 1, wherein said inhibitor is administered intramuscularly, intravenously, or subcutaneously.

4. The method of claim 1, wherein said inhibitor is administered orally.

5. The method of claim 1, wherein said inhibitor is formulated in a sustained release pill.

6. The method of claim 1, wherein said inhibitor is administered using continuous infusion or a sustained release device.

7. The method of claim 6, wherein said sustained release device is a pump or a biodegradable polymer.

8. The method of claim 1, wherein said time period is at least about three days.

9. The method of claim 1, wherein said time period is at least about one week.

10. The method of claim 1, wherein said time period is at least about two weeks.

11. The method of claim 1, wherein said time period is at least about one month.

12. The method of claim 1, wherein the concentration of said inhibitor in the blood of said human does not vary by more than about ±20% from the average concentration of said inhibitor in the blood of said human.

13. The method of claim 1, wherein the concentration of said inhibitor in the blood of said human does not vary by more than about ±15% from the average concentration of said inhibitor in the blood of said human.

14. A method of treating cancer in a human in need of such treatment, comprising administering systemically to said human a therapeutically effective amount of a small molecule hedgehog pathway inhibitor, wherein said inhibitor is present in the blood of said human at a concentration that is maintained above a threshold concentration, and remains at or below the maximum tolerated dose of said inhibitor in a human for a time period of at least about one day.

15. The method of claim 14, wherein said inhibitor is a steroidal alkaloid.

16. The method of claim 14, wherein said inhibitor is administered intramuscularly, intravenously, or subcutaneously.

17. The method of claim 14, wherein said inhibitor is administered orally.

18. The method of claim 14, wherein said inhibitor is formulated in a sustained release pill.

19. The method of claim 14, wherein said inhibitor is administered using continuous infusion or a sustained release device.

20. The method of claim 19, wherein said sustained release device is a pump or a biodegradable polymer.

21. The method of claim 14, wherein said inhibitor is present in the blood at a concentration of about two times said threshold concentration.

22. The method of claim 14, wherein said inhibitor is present in the blood at a concentration of about five times said threshold concentration.

23. The method of claim 14, wherein said inhibitor is present in the blood at a concentration of about ten times said threshold concentration.

24. The method of claim 14, wherein said inhibitor is present in the blood at a concentration of about twenty times said threshold concentration.

25. The method of claim 14, wherein said time period is at least about three days.

26. The method of claim 14, wherein said time period is at least about one week.

27. The method of claim 14, wherein said time period is at least about two weeks.

28. The method of claim 14, wherein said time period is at least about one month.

29. The method of claim 1 or 14, wherein the small molecule hedgehog pathway inhibitor is selected from the group consisting of: 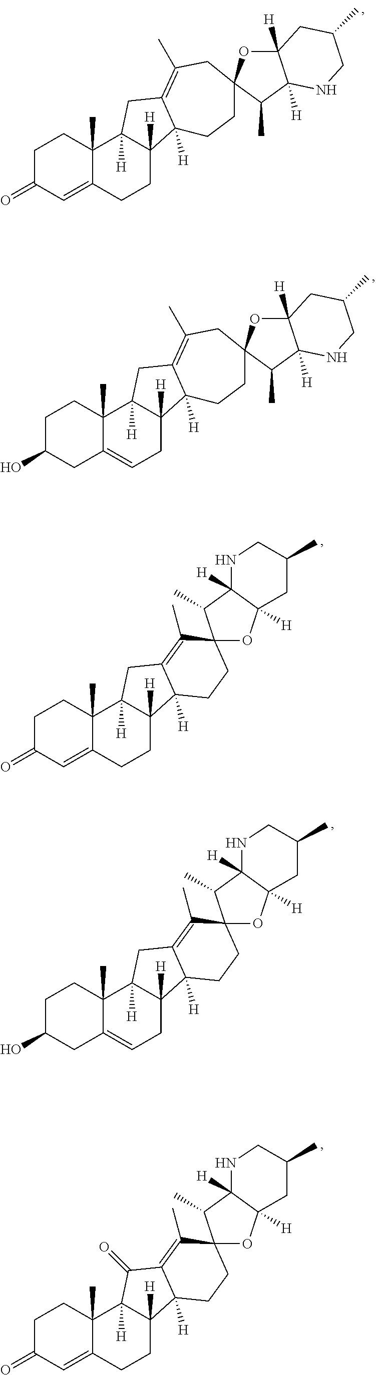 [chemical structure]
